(19) United States
(12) Patent Application Publication
(10) Pub. No.: US 2025/0076296 A1
(43) Pub. Date: Mar. 6, 2025

(54) REAGENTS AND METHODS FOR PREPARING ROBUST MESENCHYMAL STROMAL CELLS FOR THERAPY

(71) Applicant: Wisconsin Alumni Research Foundation, Madison, WI (US)

(72) Inventor: Wan-ju Li, Madison, WI (US)

(73) Assignee: Wisconsin Alumni Research Foundation, Madison, WI (US)

(21) Appl. No.: 18/611,426

(22) Filed: Mar. 20, 2024

Related U.S. Application Data
(60) Provisional application No. 63/453,341, filed on Mar. 20, 2023.

(51) Int. Cl. G01N 33/569; A61K 35/28; C12N 5/0775; G01N 33/58; G01N 33/68

(52) U.S. Cl. CPC G01N 33/56966; A61K 35/28; C12N 5/0668; G01N 33/58; G01N 33/6854

(57) ABSTRACT

The present disclosure provides reagents and methods for selecting MSCs having advantageous properties for therapeutic applications, particularly those related to cellular aging and producing MSCs of appropriate and sufficient robustness.

### PRIORITY CLAIM

This application claims priority to U.S. provisional application No. 63/453,351, filed on Mar. 20, 2023, incorporated herein by reference in its entirety.

### STATEMENT REGARDING FEDERALLY SPONSORED RESEARCH

This invention was made with government support under AR064803 awarded by the National Institutes of Health. The government has certain rights in the invention.

### INCORPORATION BY REFERENCE OF SEQUENCE LISTING PROVIDED ELECTRONICALLY

This application contains a Sequence Listing submitted as an electronic text file named “22-0996-US_SequenceListing_ST26.xml” having a size of 4,649 bytes, and created on Mar. 19, 2024. The information contained in this electronic file is hereby incorporated by reference in its entirety.

### BACKGROUND OF THE DISCLOSURE

Mesenchymal stem/stromal cells (MSCs) are multipotent cells capable of giving rise to fibroblast-like colonies (CFU-F) (Friedenstein et al., 1974, Exp. Hematol. 2:83-92). These cells can differentiate into bone, cartilage, muscle, tendons, connective tissue and stromal cells (Battula et al., 2009, Haematologica 94:173-184). These cells can be characterized by cell surface marker expression patterns; the cells express CD29, CD73, CD90, CD105, CD106, CD140b, and CD166, but do not express CD31, CD45, CD34, CD133, and class II MHC (Pittenger et al., 1999, Science 284:143-147). They can be used therapeutically for replacing damaged tissues, either autologously or allogeneically. Although arising naturally in tissues including but not limited to bone marrow, synovium, fat, and blood, using cells from these sources has been found to be disadvantageous, due to hematopoictic cell contamination and cellular heterogeneity (Seshi et al., 2000, Blood Cells Mol. Dis. 26:234-246).

A major challenge of MSC therapies is that their outcomes are not consistent, largely because the quality of MSCs varies between donors. The characteristics and activity of MSCs depend on the donor's age and health, wherein MSCs from aged or unhealthy donors are less capable of proliferation and differentiation than those from young healthy donors. Furthermore, the practice of MSC expansion through cell culture aggravates the problem of MSC aging.

Thus, there is a need in the art for reagents and methods for selecting MSCs having advantageous properties for therapeutic applications, particularly those related to cellular aging and producing MSCs of appropriate and sufficient robustness, proliferative capacity, and ability to differentiate into appropriate tissues in vivo.

### SUMMARY OF THE DISCLOSURE

Provided herein are reagents and methods for selecting MSCs, in particular human MSCs, having advantageous properties for therapeutic applications, particularly those related to cellular aging and producing MSCs of appropriate and sufficient robustness. In particular this invention provides MSCs having reduced aging profiles by cell sorting to select for cells not expressing SUSD2 on their cell surface.

Accordingly, provided herein are methods for separating a robust MSC subpopulation in a population of MSCs of variable robustness for use in stem cell therapies, the method comprising sorting the cells based on a lack of cell surface expression of SUSD2, wherein cells expressing SUSD2 are separated from cells that do not express SUSD2. In certain embodiments the cells are contacted with a detectably labeled specific binding agent for SUSD2 prior to being sorted. In particular embodiments the specific binding agent is an antibody or antigen-binding fragment thereof that specifically binds SUSD2. Advantageously, MSCs used in the methods disclosed herein are obtained from bone marrow, fat, synovium, synovial fluid, blood, periosteum, and other tissues that are important for making and repairing musculoskeletal tissues, such as cartilage, bone, tendon, ligament, and fat.

Alternative embodiments of these methods include further sorting the cells for cell surface expression of EPCAM. In certain embodiments, the cells are contacted with a detectably labeled specific binding agent for EPCAM prior to being sorted. In particular embodiments of these methods the specific binding agent is an antibody or antigen-binding fragment thereof that specifically binds EPCAM.

Advantageously, wherein the cells are sorted for expression of EPCAM and for lack of expression of SUSD2 the detectable label for the specific binding agent, in particular an antibody or antigen-binding fragment thereof specific for binding EPCAM is a different detectable label than for the specific binding agent, in particular an antibody or antigen-binding fragment thereof specific for binding SUSD2. In some embodiments of these methods the cells are sorted for lack of SUSD2 cell surface expression and presence of EPCAM cell surface expression sequentially, whereas in other embodiments the cells are sorted for lack of SUSD2 cell surface expression and presence of EPCAM cell surface expression simultaneously.

In certain specific methods provided herein the detectable label is a magnetic label and the cells are sorted by magnetic cell sorting while in alternative methods the detectable label is a fluorescent label and the cells are sorted by fluorescence-activated cell sorting methods (FACS).

Further provided herein are isolated populations of robust mesenchymal stem cells wherein the cells comprising the population do not express detectable amounts of SUSD2.

Additionally provided are isolated populations of robust mesenchymal stem cells wherein the cells comprising the population express detectable amounts of EPCAM at their cell surface and that do not express detectable amounts of SUSD2.

Also provided herein are methods for enriching from a population of mesenchymal stem cells those cells expressing EPCAM by contacting cells in the population with a detectably labeled specific binding agent for EPCAM and separating the cells thereby. In certain embodiments, the cells are contacted with a detectably labeled specific binding agent for EPCAM prior to being sorted. In particular embodiments of these methods the specific binding agent is an antibody or antigen-binding fragment thereof that specifically binds EPCAM. In additional embodiments, cells expressing EPCAM at the cell surface produced by these methods are further contacted with a detectably labeled specific binding agent that binds to SUSD2 and separating these cells from the cells produced by these methods.

In certain specific methods provided herein the detectable label is a magnetic label and the cells are sorted by magnetic cell sorting while in alternative methods the detectable label is a fluorescent label and the cells are sorted by fluorescence-activated cell sorting methods (FACS).

Further provided herein are methods for enriching from a population of mesenchymal stem cells those cells that do not express SUSD2 by contacting cells in the population with a detectably labeled specific binding agent for SUSD2 and separating the cells to exclude SUSD2-expressing cells. In certain embodiments the cells are contacted with a detectably labeled specific binding agent for SUSD2 prior to being sorted. In particular embodiments the specific binding agent is an antibody or antigen-binding fragment thereof that specifically binds SUSD2. In advantageous embodiments the detectable label is a magnetic label and the cells are sorted by magnetic cell sorting.

Provided herein also are methods for administering a population of robust mesenchymal stem cells to a patient in need thereof, wherein the population is a population not expressing SUSD2 at the cell surface, formulated at a therapeutically effective number of cells in an appropriate carrier. In alternative embodiments the cells further express EPCAM at the cell surface.

Further provided herein are methods for identifying compounds that produce upon contact or culture with MSCs a population of such MSCs having reduced expression of SUSD2 at the cell surface. Also provided are methods for administering to a patient in need thereof a therapeutically effective amounts of a compound that reduces in MSCs or selects for MSCs having reduced expression of SUSD2 at the cell surface for rejuvenation of certain tissues, including but not limited to musculoskeletal tissues, such as cartilage, bone, tendon, ligament, and fat in situ. Further provided are methods for rejuvenating MSC-comprising tissues in a patient in need thereof, comprising isolating MSCs from a tissue in the patient, treating the isolated MSCs with an MSC-rejuvenating amount of a compound identified as disclosed herein that produces reduced expression of SUSD2 at the MSC surface, and re-introducing into the tissue in the patient a tissue-rejuvenating amount of the rejuvenated MSCs.

These and other features, objects, and advantages of the present invention will become better understood from the description that follows. In the description, reference is made to the accompanying drawings, which form a part hereof and in which there is shown by way of illustration, not limitation, embodiments of the invention. The description of preferred embodiments is not intended to limit the invention to cover all modifications, equivalents, and alternatives. Reference should therefore be made to the claims recited herein for interpreting the scope of the invention.

### BRIEF DESCRIPTION OF THE DRAWINGS

The disclosure will be better understood and features, aspects, and advantages other than those set forth above will become apparent when consideration is given to the following detailed description thereof. Such detailed description refers to the following drawings.

FIG. 1 is a bar graph showing statistically significant differential expression of nine selected cell surface markers in MSCs isolated from human synovial fluid (SF-MSCs) or MSCs derived from human induced pluripotent stem cells (iPSCs) reprogrammed from the SF-MSCs (iPSC-MSCs).

FIG. 2 is a bar graph of differential expression of seven selected cell surface markers in young and aged SF-MSCs showing statistical significance for FCGR2A, SUSD2, and EPCAM, wherein SUSD2 and FCGR2A expression is increased and EPCAM expression is decreased in aged compared with young SF-MSCs.

FIG. 3 is a bar graph showing differential expression of seven selected cell surface markers in SF-MSCs transfected by short interfering RNA (siRNA) species specific for transcription factor regulator GATA6 (si-GATA6) or “scrambled” siRNA having no specificity for GATA6 (si-SCR), wherein statistically significant changes in expression were observed for CD6, EPCAM, and SUSD2, indicating their roles in regulating GATA6-mediated MSC aging. Since CD6 was not shown differentially expressed between young and aged MSCs in FIG. 2, the molecule was excluded as a potential marker. Particularly EPCAM and SUSD2 are identified as potential cell surface markers to differentiate young from aged MSCs.

FIG. 4 are bar graphs showing expression of p53, p21^{Cip1}, and Cdk1 in mouse periosteal MSCs transfected by siRNA specific for Epcam (si-Epcam) or scrambled siRNA having no specificity for Epcam (si-SCR). The result of loss-of-function assay shows that the depletion of Epcam leads to increased levels of cell cycle inhibitors (p53 and p21) and reduced levels of the cell cycle regulator (Cdk1), suggesting that Epcam promotes cell cycle progression, positioning it as an anti-aging marker in MSCs.

FIG. 5 are bar graphs showing expression of p53, p21^{Cip1}, and Cdk1 in mouse periosteal MSCs transfected by siRNA specific for Susd2 (si-Susd2) or scrambled siRNA having no specificity for Susd2 (si-SCR). The result of loss-of-function assay shows that the depletion of Susd2 leads to reduced levels of cell cycle inhibitors (p53 and p21) and increased levels of the cell cycle regulator (Cdk1), suggesting that Susd2 prevents cell cycle progression, positioning it as an aging marker in MSCs.

FIG. 6 is a flow cytometry plot of human bone marrow-derived MSCs (BM-MSCs) labeled with antibodies specific for EPCAM (CD326) or SUSD2 conjugated to antibody-specific fluorescent dyes. A relatively larger population is SUSD2⁺ MSCs while a much smaller population is EPCAM⁺ MSCs.

FIG. 7 is a flow cytometry plot of BM-MSCs from early (P4) and late (P9) cell passage culture as in vitro-induced young and aged/senescent MSCs, respectively, showing the distribution of cells bound with SUSD2-specific antibody as SUSD2⁺ MSCs or that of cells without bound SUSD2 antibody as SUSD2⁻ MSCs. The percentage of SUSD2⁺ MSCs increases from 65% to 71% with cell passage, indicating SUSD2 is a viable marker for identifying aged MSCs.

FIG. 8 are bar graphs showing p53 and GATA6 expression in the SUSD2⁺ or SUSD2⁻ BM-MSC subpopulation at P4 and P9. SUSD2⁺ MSCs at both P4 and P9 expressed higher levels of p53 and GATA6 than SUSD2⁻ MSCs, indicating the positive association between the pro-aging regulators and the aging cell surface markers.

FIG. 9 are bar graphs showing expression of the bone-associated markers alkaline phosphatase (ALP) and osteocalcin (OC) and the fat-associated markers C/EBP3 and lipoprotein lipase (LPL) in the SUSD2⁺ or SUSD2⁻ BM-MSC subpopulations at P4 and P9. SUSD2⁺ MSCs at both P4 and P9 lean toward adipogenic differentiation and away from osteogenic differentiation compared to SUSD2⁻ MSCs. The results are consistent with the established knowledge that aged MSCs are prone to turning into fat cells rather than bone cells. See, e.g., Choudhery et al., 2014, J Transl. Med. 12:1-14.

### DETAILED DESCRIPTION OF THE DISCLOSURE

The present disclosure is based on the properties of MSCs, wherein cells with reduced or no expression of SUSD2 have a phenotype associated with reduced cellular aging and robustness in therapeutic applications.

For the purposes of promoting an understanding of the principles of the disclosure, reference will now be made to embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

### Definitions

As used in the specification, articles “a” and “an” are used herein to refer to one or to more than one (i.e., at least one) of the grammatical object of the article. By way of example, “an element” means at least one element and can include more than one element.

“About” is used to provide flexibility to a numerical range endpoint by providing that a given value can be “slightly above” or “slightly below” the endpoint without affecting the desired result. The term “about” in association with a numerical value means that the numerical value can vary by plus or minus 5% or less of the numerical value.

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements, or steps but not the exclusion of any other integer or step or group of integers or steps.

As used herein, “and/or” refers to and encompasses any and all possible combinations of one or more of the associated listed items, as well as the lack of combinations where interpreted in the alternative (“or”).

Recitation of ranges of values herein are merely intended to serve as a succinct method of referring individually to each separate value falling within the range, unless otherwise indicated herein. Furthermore, each separate value is incorporated into the specification as if it were individually recited herein. For example, if a range is stated as 1 to 50, it is intended that values such as 2 to 4, 10 to 30, or 1 to 3, etc., are expressly enumerated in this disclosure. These are only examples of what is specifically intended, and all possible combinations of numerical values between and including the lowest value and the highest value enumerated are to be considered to be expressly stated in this disclosure.

Unless otherwise defined, all technical terms used herein have the same meaning as commonly understood by a person of ordinary skill in the art to which this disclosure belongs.

The term “contacting” includes the physical contact of at least one substance to another substance.

As used herein, “treatment” refers to the clinical intervention made in response to a disease, disorder, or physiological condition of the subject or to which a subject can be susceptible. The aim of treatment includes the alleviation or prevention of symptoms, slowing or stopping the progression or worsening of a disease, disorder, or condition and/or the remission of the disease, disorder, or condition.

The terms “effective amount” or “therapeutically effective amount” refer to an amount sufficient to effect beneficial or desirable biological and/or clinical results. In other words, a “therapeutically effective” amount is an amount that will provide some alleviation, mitigation, or decrease in at least one clinical symptom in the subject.

The terms “express” or “expression” refer to transcription and translation of a nucleic acid coding sequence resulting in production of the encoded polypeptide. “Express” or “expression” also refers to antigens that are expressed on cell surfaces.

As used herein, the term “subject” refers to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. The subject can be a human patient that is at risk for, or suffering from, diseases including but not limited to heart disease, musculoskeletal disease such as traumatic diseases like bone fracture and chronic diseases such as osteoarthritis, as well as heart disease and infectious diseases. The human subject can be of any age (e.g., an infant, child, or adult).

Any appropriate method can be used to detect expression of biological markers characteristic of cell types described herein. For example, the presence or absence of one or more biological markers can be detected using, for example, RNA sequencing (e.g., RNA-seq), immunohistochemistry, polymerase chain reaction, quantitative real time-polymerase chain reaction (qRT-PCR), or other technique that detects or measures gene expression. RNA-seq is a high-throughput sequencing technology that provides a genome-wide assessment of the RNA content of an organism, tissue, or cell. Alternatively, or additionally, one can detect the presence or absence of, or measure the level of, one or more biological markers using, for example, fluorescence in situ hybridization (FISH; see WO98/45479 published October 1998), western blotting, Southern blotting, Northern blotting, or PCR techniques, such as reverse transcription (RT)-PCR and qRT-PCR. In exemplary embodiments, a cell population obtained according to a method provided herein is evaluated for expression (or the absence thereof) of biological markers of HPCs such as CD34, CD45, CD43, CD49f, and CD90. Quantitative methods for evaluating expression of markers at the protein level in cell populations are also known in the art.

In some embodiments, cell surface marker sorting techniques used in the practice of the methods disclosed herein include, but are not limited to, fluorescent activated cell sorting (FACS), magnetic activated cell sorting (MACS), immunoprecipitation, immunodensity cell isolation, and centrifugation.

In specific embodiments, the cell surface marker sorting technique is FACS or MACS.

In some embodiments, cells useful in the practice of this invention are pluripotent stem cells which can be an embryonic stem cell or an induced pluripotent stem cell.

Human pluripotent stem cells (hPSCs), either embryonic or induced, provide access to the earliest stages of human development and offer a platform on which to derive a large number of tissue-specific cells for cellular therapy and tissue engineering.

As used herein, the term “mesoderm cell” or “mesodermal cell” refers to a cell having mesoderm-specific gene expression, capable of differentiating into a mesodermal lineage such as bone, muscle such as cardiac muscle, skeletal muscle, and smooth muscle (e.g., of the gut), connective tissue such as the dermis and cartilage, kidneys, the urogenital system, blood or hematopoietic cells, heart, and vasculature. Mesoderm-specific biomarkers include Brachyury (T). Culturing can take place on any appropriate surface (e.g., in two-dimensional or three-dimensional culture).

Medium and substrate conditions for culturing pluripotent stem cells, as used in the methods described herein, are well known in the art. In some cases, pluripotent stem cells to be differentiated according to the methods disclosed herein are cultured in mTESR-1® medium (StemCell Technologies, Inc., Vancouver, British Columbia.), E8 medium, or Essential 8® medium (Life Technologies, Inc.) on a MATRIGEL™ substrate (BD Biosciences, NJ) according to the manufacturer's protocol or on a Corning™ Synthemax™ surface.

Human pluripotent stem cells (e.g., human ESCs or iPS cells) can be cultured in the absence of a feeder layer (e.g., a fibroblast feeder layer), a conditioned medium, or a culture medium comprising poorly defined or undefined components.

As used herein, the terms “chemically-defined medium” and “chemically-defined culture medium” also refer to a culture medium containing formulations of fully disclosed or identifiable ingredients, the precise quantities of which are known or identifiable and can be controlled individually. As such, a culture medium is not chemically-defined if (1) the chemical and structural identity of all medium ingredients is not known, (2) the medium contains unknown quantities of any ingredients, or (3) both. Standardizing culture conditions by using a chemically-defined culture medium minimizes the potential for lot-to-lot or batch-to-batch variations in materials to which the cells are exposed during cell culture. Accordingly, the effects of various differentiation factors are more predictable when added to cells and tissues cultured under chemically-defined conditions.

As used herein, the term “serum-free” refers to cell culture materials that do not contain serum or serum replacement, or that contains essentially no serum or serum replacement. For example, an essentially serum-free medium can contain less than about 1%, 0.9%, 0.8%, 0.7%, 0.6%, 0.5%, 0.4%, 0.3%, 0.2%, or 0.1% serum. “Serum free” also refers to culture components free of serum obtained from animal (e.g., fetal bovine) blood or animal-derived materials, which is important to reduce or eliminate the potential for cross-species viral or prion transmission. For avoidance of doubt, serum-containing medium is not chemically-defined.

As used herein, “feeder-free” refers to culture conditions that are substantially free of a cell feeder layer. Cells grown under feeder-free conditions can be grown on a substrate, such as a chemically-defined substrate, and/or grown as an adherent culture. Suitable chemically-defined substrates include vitronectin.

As used herein, “pluripotent stem cells” appropriate for use according to a method of the invention are cells having the capacity to differentiate into cells of all three germ layers. Suitable pluripotent cells for use herein include human embryonic stem cells (hESCs) and human induced pluripotent stem (iPS) cells. As used herein, “embryonic stem cells” or “ESCs” mean a pluripotent cell or population of pluripotent cells derived from an inner cell mass of a blastocyst. Scc Thomson et al., Science 282:1145-1147 (1998). These cells can express Oct-4, SSEA-3, SSEA-4, TRA-1-60, and TRA-1-81. Pluripotent stem cells appear as compact colonies comprising cells having a high nucleus to cytoplasm ratio and prominent nucleolus. ESCs are commercially available from sources such as WiCell Research Institute (Madison, WI.).

As used herein, “induced pluripotent stem cells” or “iPS cells” refers to pluripotent cell or population of pluripotent cells that can vary with respect to their differentiated somatic cell of origin, that can vary with respect to a specific set of potency-determining factors and that can vary with respect to culture conditions used to isolate them, but nonetheless are substantially genetically identical to their respective differentiated somatic cell of origin and display characteristics similar to higher potency cells, such as ESCs, as described herein. Sec, e.g., Yu et al., Science 318:1917-1920 (2007).

Induced pluripotent stem cells exhibit morphological properties (e.g., round shape, large nucleoli, and scant cytoplasm) and growth properties (e.g., doubling time of about seventeen to eighteen hours) akin to ESCs. In addition, iPS cells express pluripotent cell-specific markers (e.g., Oct-4, SSEA-3, SSEA-4, Tra-1-60 or Tra-1-81, but not SSEA-1). Induced pluripotent stem cells, however, are not immediately derived from embryos. As used herein, “not immediately derived from embryos” means that the starting cell type for producing iPS cells is a non-pluripotent cell, such as a multipotent cell or terminally differentiated cell, such as somatic cells obtained from a post-natal individual.

As used herein the term “mesenchymal stem cell” or “mesenchymal stromal cells” will be understood to mean multipotent cells found in bone marrow, fat, synovium, synovial fluid, blood, periosteum, and may other tissues that are important for making and repairing musculoskeletal tissues, such as cartilage, bone, tendon, ligament, and fat. With age and disease, MSCs in vivo predominantly convert into lipid-accumulating fat cells or become senescent.

As used herein, the terms “robust” and “robustness” will be understood by the skilled worker to characterize cells described thereby as having long-term albeit not unlimited proliferative capacity, low or no cellular senescence, low or no expression of aging hallmarks (see Lopez-Otin et al., 2013, Cell 153:1194-1217), sustained multilineage differentiation capacity, and other characteristics of cells from animals prior to cell aging commences.

### Pharmaceutical Formulations and Methods of Treatment

In exemplary embodiments, the methods provided herein are conducted in accordance with Good Manufacturing Practices (GMPs), Good Tissue Practices (GTPs), and Good Laboratory Practices (GLPs). Reagents comprising animal derived components are not used, and all reagents are purchased from sources that are GMP-compliant. In the context of clinical manufacturing of a cell therapy product, GTPs govern donor consent, traceability, and infectious disease screening, whereas the GMP is relevant to the facility, processes, testing, and practices to produce a consistently safe and effective product for human use. See Lu et al. Stem Cells 27:2126-2135 (2009). Where appropriate, oversight of patient protocols by agencies and institutional panels is envisioned to ensure that informed consent is obtained; safety, bioactivity, appropriate dosage, and efficacy of products are studied in phases; results are statistically significant; and ethical guidelines are followed.

Provided herein are populations of MSCs characterized by reduced or absent expression of SUSD2 on the cell surface; such MSCs are understood to have a more robust, “younger” phenotype and thus are advantageous for administration to an individual or patient in need of MSCs and particularly for robust, “youthful” MSCs. As disclosed herein, these methods of delivering a therapeutically effective amount of robust MSCs as defined herein can provide rejuvenation of certain tissues, including but not limited to musculoskeletal tissues, such as cartilage, bone, tendon, ligament, and fat.

In alternative embodiments a therapeutically effective amount of a compound that reduces in MSCs or selects for MSCs having reduced expression of SUSD2 at the cell surface are administered to a patient in need thereof for rejuvenation of certain tissues, including but not limited to musculoskeletal tissues, such as cartilage, bone, tendon, ligament, and fat in situ.

Alternatively, rejuvenated MSCs can be raised from MSCs isolated from a tissue or tissues in a patient by treating such isolated MSCs with an MSC-rejuvenating amount of a compound identified as disclosed herein or culturing the isolated MSCs under conditions or in a medium that produces reduced expression of SUSD2 at the MSC surface, and re-introducing into the tissue or tissues in the patient a tissue-rejuvenating amount of the rejuvenated MSCs optionally after sorting cells on the basis of cell surface SUSD2 expression as disclosed herein.

Any appropriate dosage of the MSC rejuvenating compounds or rejuvenated MSCs produced by treatment of such compounds can be used for a therapeutic method provided herein.

After administering the cells into the subject, the effect of the treatment method can be evaluated, if desired, using any appropriate method known to practitioners in the art. The treatment can be repeated as needed or required.

Various exemplary embodiments of compositions and methods according to this invention are now described in the following non-limiting Examples. The Examples are offered for illustrative purposes only and are not intended to limit the scope of the present invention in any way. Indeed, various modifications of the invention in addition to those shown and described herein will become apparent to those skilled in the art from the foregoing description and the following examples and fall within the scope of the appended claims.

### EXAMPLES

The Examples set forth herein incorporate and rely on certain experimental and preparatory methods and techniques performed as exemplified herein.

### Materials and Methods

### Isolation and Culture of Tissue-Derived MSCs

To isolate MSCs from synovial fluid (SF-MSCs), harvested synovial fluid was diluted 1:4 with phosphate-buffered saline (PBS), filtered through a 70-μm nylon filter (Becton Dickinson, Franklin Lakes, NJ, USA), and resuspended after centrifugation at about 500 g. Cells were plated in culture flasks with medium composed of low-glucose Dulbecco Modified Eagle Medium (DMEM), 10% fetal bovine serum (FBS; Atlanta Biologicals, Atlanta, GA, USA), and antibiotics. To isolate human bone marrow stem cells (BMSCs), bone marrow was collected, suspended in DMEM, and centrifuged at 1000 rpm for 5 min. After the supernatant was removed, the cell pellet was reconstituted with HBSS (Thermo Fisher Scientific, Waltham, MA, USA). In a new tube containing 20 ml of Ficoll solution (GE Healthcare, Chicago, IL, USA), the cell mixture was added and centrifuged at 500 g for 30 min before mononuclear cells were collected and seeded into tissue culture flasks or plates. Both SF-MSCs and BMSCs were cultured with complete medium and maintained in an incubator at 37° C. in a humidified 5% CO₂ atmosphere. After reaching 70 to 80% confluency, the cells were released from their attachment to the plates using 0.05% trypsin/EDTA (Gibco) and re-plated at a seeding density of 1,000 cells/cm². Culture medium was replaced every 3 days. To isolate mouse BMSCs, bone marrow was flushed out of harvested tibiac and femurs with complete medium composed of DMEM, 10% FBS, and antibiotics. After filtered through a 70-μm mesh, cells were seeded in a T75 flask for analysis.

Culture of Transgene-Free iPSCs

SF-MSCs transfected with episomal plasmids encoding OCT4, SOX2, KLF4, c-MYC, NANOG, and LIN28 were plated in 2 Matrigel-coated 6-well plates containing Essential 8 (E8) medium and 1 M cortisone. Culture medium was changed daily and replaced with E7 medium (E8 without TGFB1) when cells reached 20-30% confluence. After 3 weeks, cell colonies positive for TRA-1-60 staining and negative for CD44 were picked under microscope and each colony was individually transferred to a well of Matrigel-coated 24-well plates with E8 medium for expansion and passaging afterwards.

Derivation and Culture of iPSC-MSCs

iPSCs were cultured in Matrigel-coated 6-well plates with E8 medium until they reached 80% confluence, and then induced by STEMdiff™-ACF Mesenchymal Induction Medium for 4 days with daily medium change. To derive mesenchymal progenitor cells, cells were then cultured in complete MesenCult™-ACF Plus Medium for another 2 days with daily medium change. After the 2 days culture, cells were passaged, considered iPSC-MSCs at P1, and continued to be cultured with complete MesenCult™-ACF Plus Medium for another 4 passages. To maintain iPSC-MSCs, the culture medium was then switched to normal growth medium composed of DMEM, 10% FBS, and antibiotics.

### Biological Assays and Analyses

### Flow Cytometric Analysis

Cells were trypsinized and washed twice using flow cytometry staining buffer made of ice-cold PBS, 0.1% sodium azide, and 1% bovine serum albumin (Sigma-Aldrich, Burlington, MA, USA). Antibodies detecting pluripotency markers, TRA-1-60, NANOG, OCT4, SOX2, and SSEA4, and those detecting surface markers, CD73, CD90, CD105, CD45, CD31, SUSD2, and EPCAM to identify MSCs were used together with or without secondary antibodies for analysis. Detailed information of the antibodies used herein is provided in Table A. Briefly, for detection of TRA-1-60, cells were incubated with PE-conjugated mouse antibody for 30 min, washed with staining buffer 3 times to remove unbound antibody, and analyzed by flow cytometry. To detect SSEA4, cells were incubated with mouse primary antibody for 30 min, followed by incubation with Alexa Fluor 647-conjugated goat secondary antibody at 1:200 for another 30 min and then washed with staining buffer 3 times to remove unbound antibody before analysis. To detect NANOG, OCT4, and SOX2, cells were fixed and permeabilized with 1% paraformaldehyde, washed with PBS containing 0.2% Tween 20, incubated with goat or mouse primary antibodies for 30 min, followed by incubation with NL493-conjugated donkey or Alexa Fluor 647-conjugated goat secondary antibody, respectively, at 1:200 for another 30 min and then washed with staining buffer 3 times to remove unbound antibody before analysis. To identify MSCs, cells were incubated with fluorochrome-conjugated mouse antibody for 30 min, washed with staining buffer 3 times to remove unbound antibody, and analyzed by flow cytometry. Expression of surface and intracellular markers was analyzed by MACSQuant Analyzer 10 (Miltenyi Biotec, Bergisch Gladbach, Germany). Data were analyzed using the FlowJo software (TreeStar, Ashland, OR, USA).

TABLE A
Detailed information of the antibodies used herein

| Primary antibodies | Company | Catalog number |
|---|---|---|
| PE, Mouse anti-TRA-1-60 | BD Bioscience | 560193 |
| PE, Mouse anti-CD73 | BD Bioscience | 550257 |
| APC, Mouse anti-CD90 | BD Bioscience | 559869 |
| FITC, Mouse anti-CD105 | BD Bioscience | 561443 |
| PERCP-CY ™5.5, Mouse anti- | BD Bioscience | 564106 |
| CD45 | | |
| PE, Mouse anti-CD31 | BD Bioscience | 560983 |
| APC, Mouse anti-CD326 | BioLegend | 324207 |
| (EPCAM) | | |
| PE, Mouse anti-SUSD2 | Miltenyi Biotec | 130-117-794 |
| Goat anti-NANOG | R&D | SC009 |
| Goat anti-OCT4 | R&D | SC009 |
| Mouse anti-SSEA4 | R&D | SC009 |
| Mouse anti-SOX2 | R&D | SC009 |
| Rabbit anti-p53 | CST | 9282 |
| Rabbit anti-p21 Waf1/ | CST | 2947 |
| Cip1(12D1) | | |
| Rabbit anti-CDK1 (cdc2) | CST | 28439 |
| Rabbit anti-GAPDH | CST | 2118 |
| Mouse anti-FOXP1 | Novus Biologicals | NB100-65125 |
| Rabbit anti-Sonic Hedgehog | Novus Biologicals | NBP2-22139SS |
| Rabbit anti-GLI-1 | Novus Biologicals | NB600-600SS |
| Rabbit anti-GATA6 | Novus Biologicals | MAB1700 |
| Rabbit anti-Smoothened | Abcam | ab236465 |
| Rabbit anti-LC3B | R&D | MAB85582 |
| Rabbit anti-SIRT1 | CST | 9475 |
| Mouse anti-JNK1/2 | R&D | MAB2076 |
| Rabbit anti-phospho-JNK1/2 | R&D | MAB1205 |
| Secondary antibodies | Company | Catalog number |
| Alexa Fluor 647, goat | ThermoFisher | A21235 |
| anti-mouse IgG | Scientific | |
| Alexa Fluor 546, donkey | ThermoFisher | A10036 |
| anti-mouse IgG | Scientific | |
| NL493, donkey anti-goat IgG | R&D | NL003 |
| Anti-Rabbit IgG, HPR-linked | CST | 7074 |
| Anti-Mouse IgG, HPR-linked | CST | 7076 |

### Immunofluorescence Analysis

Cells grown in glass bottom dishes (MatTek, Ashland, MA, USA) were stained with TRA-1-60 (BD Biosciences) for 30 min, followed by image acquisition. For detection of NANOG, OCT4, SOX2, or SSEA4 (R&D Systems), cells were fixed with 4% paraformaldehyde, permeabilized and blocked with 0.3% Triton X-100 and 1% BSA in PBS, and stained overnight at 4° C. with primary antibody, followed by incubation with secondary antibody at 1:200 (donkey anti-goat IgG NL493 or donkey anti-mouse IgG Alexa Fluor 546, Thermo Fisher Scientific). Staining of 4, 6-Diamidino-2-phenylindole, dihydrochloride (DAPI) was performed for 10 min to visualize nuclei before imaging by a laser scanning confocal microscope (Nikon AIRS, Japan).

### Total RNA Extraction and Quantitative RT-PCR

Total RNA was extracted from cells using the Zymo Quick-RNA MicroPrep kit (Zymo Research, Irvine, CA, USA) following the manufacturer's instructions. One microgram of total RNA was used in each reaction with the High-Capacity cDNA Reverse Transcription kit (Applied Biosystems, Carlsbad, CA, USA) to synthesize cDNA. Quantitative RT-PCR was performed using the iQ SYBR Green Premix (Bio-Rad, Hercules, CA, USA) with primers detecting sex determining region Y-box 9 (SOX9), collagen type 2 (COL2), aggrecan (ACAN), peroxisome proliferator-activated receptor gamma 2 (PPARG2), lipoprotein lipase (LPL), core-binding factor subunit alpha 1 (CBFAI), alkaline phosphatase (ALP), osteocalcin (OC), telomerase reverse transcriptase (TERT), p53, p21^{CIP1}, p16^{INK4A}, GATA6, CD28, FCGR2A, CD8A, CD8B, CD6, ICAM2, MYOCD, SUSD2, EPCAM, and ubiquitin C (UBC). The 2^{-ΔCT} method was used to determine relative expression levels of a target transcript to those of UBC as an internal control.

### Cell Growth Analysis

Long-term growth of SF-MSCs and iPSC-MSCs was measured by cumulative population doublings (PDs) of the cell. To obtain values of PDs, cell numbers were determined at each passage using a hemocytometer and then PDs were calculated based on the equation PD=X+3.322 (log Y−log I) where “X” is the doubling level of the inoculum used to initiate the subculture being quantitated, “Y” is the number of cells at that point, and “I” is the number of cells used as inoculum to begin that subculture.

### Immunomodulatory Assay

To evaluate the immunomodulatory ability of MSCs, 1×10⁵ SF-MSCs or iPSC-MSCs were co-cultured with 4×10⁵ human peripheral blood mononuclear cells (PBMCs) that had been prelabeled with 0.5 μM CellTrace™ CFSE (Millipore, Billerica, MA, USA) in 12-well transwell plates. To stimulate PBMCs, 20 μg/ml of phytohacmagglutinin (PHA, Sigma-Aldrich) was added in the co-culture. After 3 days of co-culture, proliferation of labeled PBMCs was measured by MACSQuant Analyzer 10. PBMCs treated with or without PHA served as a positive or negative control, respectively.

### Assessment of Multi-Lineage Differentiation

Adipogenic, chondrogenic, and osteogenic differentiation of SF-MSCs and iPSC-MSCs was induced as previously described in Tsai et al., 2015, Cell Res & Ther. 6:88. Briefly, cells were cultured in adipogenic, chondrogenic, or osteogenic medium for differentiation. After 21 days of induction, adipogenic differentiation was analyzed by staining of Oil red O (Sigma-Aldrich). Chondrogenic differentiation was assessed by Alcian blue staining and GAGs quantification. Osteogenic differentiation was determined by staining of Alizarin red S (Rowley Biochemical, Danvers, MA, USA), calcium quantification, and cytochemical staining and quantification of ALP activity. In addition to histological and biochemical analyses, the transcript expression of fat-associated (PPARG2, LPL), cartilage-associated (SOX9, COL2, ACAN), and bone-associated markers (CBFAI, OC, ALP) was analyzed by quantitative RT-PCR following the method described above.

### Senescence-Associated β-Galactosidase (SA-β-Gal) Staining

The activity of SA-β-gal was assessed by a senescence detection kit (Dojindo Molecular Technologies, Rockville, MD, USA) following the manufacturer's instructions. Briefly, cells were fixed with 4% formaldehyde for 3 min, incubated with the SPIDER-β-gal working solution at 37 C.° for 30 min, and stained with DAPI for 10 min before imaged by a fluorescence microscope. The percentage of senescent cells was determined by the number of stained cells relative to that of total cells.

### Telomerase Activity Measurement

Telomerase activity was determined by the TRAPeze® RT Telomerase Detection kit (Millipore, Billerica, MA, USA) following the manufacturer's instructions. Cell extracts were prepared using the CHAPS lysis buffer provided in the kit and protein concentrations were measured using the BCA Protein Assay kit (Pierce, Rockford, IL, USA). One microgram of total protein from each sample was added to each reaction. Heat-treated controls from each sample were included in order to rule out false-positive signals from PCR artifacts.

### Determination of Relative Telomere Length

Relative telomere length was determined by the method described by Cawthon et al. (2002, Nucleic Acids Res. 30: e47) with minor modifications. Briefly, DNA was extracted from cells using the QIAamp DNA Mini kit (QIAGEN) following the manufacturer's instructions. Telomere length was quantified using quantitative RT-PCR by comparing the telomere repeat sequence to a single copy gene (36B4). The primer sequences of telomere and 36B4 are listed in the cited reference to be

telomere 1,
(SEQ ID NO. 1)
GGTTTTTGAGGGTGAGGGTGAGGGTGAGGGTGAGGGT;
telomere 2,
(SEQ ID NO. 2)
TCCCGACTATCCCTATCCCTATCCCTATCCCTATCC-CTA;
36B4u gene,
(SEQ ID NO. 3)
CAGCAAGTGGGAAGGTGTAATCC;
36B4d gene,
(SEQ ID NO. 4)
CCCATTCTATCATCAACGGGTACAA.

The reaction mix consisted of iQ SYBR Green Premix (Bio-Rad), forward and reverse primers, and 35 ng of DNA per reaction. In order to generate a standard curve, serial dilutions of reference samples for telomere and 36B4 PCR reactions were included. With quantitative RT-PCR results, a plot of standard curve showing Ct versus logarithm of the amount of input reference DNA was constructed and the telomere repeat copy number (T) and single control gene copy number(S) in each sample was determined by comparison to the standard curve. The relative telomere length of each sample was measured by calculating the ratio of T/S.

### Protein Extraction and Western Blotting Analysis

Cells were lysed in RIPA buffer composed of 50 mM Tris-HCl (pH 7.5), 0.25% Na-deoxycholate, 1% Nonidet P-40, 150 mM NaCl, 1 mM EDTA, and complete protease inhibitor cocktail (Roche, Indianapolis, IN, USA) and then centrifuged at 14,000 rpm for 10 min to collect supernatant. Protein concentration in the supernatant was measured using the BCA Protein Assay kit (Pierce). A 20-μg protein sample was loaded into each lane of a 10% polyacrylamide gel (Bio-Rad) for electrophoresis, and separated proteins were then transferred from the gel onto a nitrocellulose membrane (Bio-Rad). The membrane was incubated with primary antibodies against p53, p21^{CIP1}, CDK1 (Cell Signaling Technology, Danvers, MA, USA), SHH, GLI1, GATA6, FOXP1 (Novus Biologicals, Centennial, CO, USA), and SMO (Abcam, Cambridge, MA, USA) in blocking solution composed of Tris-buffered saline containing 5% nonfat milk (Bio-Rad) and 0.1% Tween 20 (Sigma-Aldrich) overnight at 4° C. After removing unbound antibodies, the membrane was incubated with horseradish peroxidase-linked secondary antibody (Cell Signaling Technology) in the blocking solution for 1 h at room temperature. Immuno-detected protein bands on the membrane were visualized using the SuperSignal West Pico Chemiluminescent Substrate (Pierce), and then documented by the Kodak Image Station 4000R Pro system (Kodak, Rochester, NY, USA).

### Co-Immunoprecipitation (Co-IP)

Co-IP was performed to identify protein-protein interactions in iPSC-MSCs. Cell extracts using lysis buffer were prepared as described above. Lysates were incubated overnight at 4° C. with anti-p53, anti-FOXP1, or IgG control (Cell Signaling Technology, Danvers, MA, USA). Antibody-antigen complexes were then incubated with Protein A/G PLUS-Agarose (Santa Cruz Biotechnology, Dallas, Texas, USA) for 2 h before washed with lysis buffer 3 times, resuspended in SDS gel loading buffer, and boiled for 5 min. The boiled samples were loaded on a 10% SDS-polyacrylamide gel for western blot analysis.

### RNA Interference-Mediated Gene Silencing

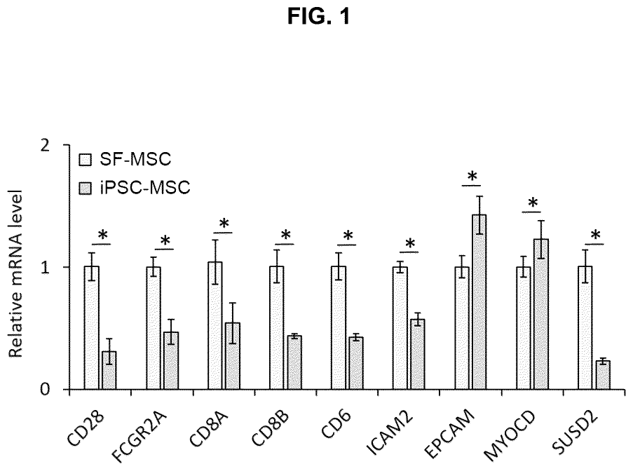

All synthetic small interfering RNA (siRNA) designed to target the nucleotide sequence for silencing CD28, FCGR2A, CD8A, CD8B, CD6, ICAM2, MYOCD, GATA6, SUSD2, and EPCAM along with scrambled siRNA were purchased from QIAGEN. Cells were transfected individually with each of the siRNAs or scrambled siRNA using the GenMute siRNA Transfection Reagent (SignaGen Laboratories, Gaithersburg, MD) following the manufacturer's instructions. To evaluate effects of gene knockdown, RNA and protein were harvested from cells 48 hours post-transfection for analysis. Silenced genes are set forth in FIG. 1 herein.

### RNA-Sequencing (RNA-Seq) and Data Processing and Analysis

Cells were lysed in Trizol and stored at −80° C. until RNA isolation. Total RNA was extracted using the Direct-zol™ RNA kit (Zymo Research) following the manufacturer's instructions and quantified and quality-checked using the NanoDrop 1000™ Spectrophotometer (Thermo Fisher Scientific) and the BioAnalyzer RNA 6000 Nano kit (Agilent Technologies). The cDNA library was prepared using the NEXTflex™ Rapid Directional RNA Sequencing kit and NEXTflex™ RNA-Seq Barcodes-24 kit (BioO Scientific) following the manufacturer's instructions. The final product was assessed for size distribution using the Bioanalyzer DNA High Sensitivity kit (Agilent Technologies) and for concentration using the Kapa library quantification kit (Kapa Biosystems). Libraries were pooled equimolarily, followed by on-board cluster generation on a Rapid Run single-end flow cell and subsequent 50 cycles sequencing (v3 sequencing kit) according to the manufacturer's instructions (HiSeq 2500, Illumina). Raw sequencing reads in BCL format were processed through CASAVA-1.8.2 for FASTQ conversion and demultiplexing. For bioinformatic analysis, reads (strand-specific, single-end 50 bp) were aligned to the human genome (GRCh38/Hg38) using the Tophat suite (v.2.1.1) with Ensembl release 88 annotations to guide transcript assembly. Cuffmerge was used to merge transcript assemblies from individual samples into one master file for counting. Using this master file (after removing unstranded transcripts), gene-level counts were done with HTSeq-count (v.0.8.0) with default values except for “-stranded reverse”. Counts were loaded into R and analyzed with DESeq2. Data were fitted to both donor and cell type. Pairwise contrasts were extracted and genes with adjusted P-value <0.05 and fold change >2 were deemed to be differentially expressed (DE). DESeq2 rlog (blind=FALSE) was used to log-normalize counts for heat maps and other plots. Heat maps were generated with the R package pheatmap (v.1.0.8). Singular value decomposition (svd) was used with centered log 2 counts for PCA plots. Gene set enrichment analysis (GSEA v.2.2.2) with Hallmark gene signatures (Huang et al., 2009, Nat Protoc. 4:44-57) was run on genes pre-ranked by log 2 fold change. DAVID (v.6.8) was used for functional enrichment analysis against all human genes as background using a subset of annotations: Gene Ontology (BP Direct, CC Direct, MF Direct), Pathways (KEGG, Reactome), Protein Interactions (UCSC TFBS), and Tissue Expression (Up Tissue).

### Measurement of Mitochondrial Membrane Potential (MMP)

The MMP of cells was assessed by the MitoProbe™ JC-1 Assay kit (Thermo Fisher Scientific,) following the manufacturer's instructions to determine mitochondrial health. Briefly, cells were harvested, suspended in 1 ml warm PBS, and stained with 2 μM JC-1 dye for 30 min in dark at 37° C. Cells were then washed once, resuspend in 0.5 ml PBS, and analyzed by flow cytometer. Data were analyzed using the FlowJo software (TreeStar, Ashland, OR, USA). Accumulation of JC-1 dye is dependent on the membrane potential of mitochondria, indicated by a fluorescence emission shift from the wavelength at 530 nm (green) to that at 588 nm (red). MMP was measured by calculating the ratio of red to green fluorescence intensity (Gunjan et al., 2016, Apoptosis 21:955-964).

### Quantification of DNA Methylation and Hydroxymethylation

Genomic DNA was extracted from cells using the Zymo Quick-DNA MicroPrep kit per manufacturer's instructions. Genomic DNA methylation and hydroxymethylation levels were determined by the MethylFlash Global DNA methylation (5-mC) ELISA Easy kit and the MethylFlash Global DNA Hydroxymethylation (5-hmC) ELISA Easy kit (Epigentek, Farmingdale, NY, USA) following the manufacturer's protocols.

### Example 1

### Identification of Cell Surface Markers for Age-Related Markers

To determine useful cell surface markers for mesenchymal stem cells (MSCs) having young phenotypes (including rejuvenated iPSC-MSCs and tissue-derived MSCs at early cell passages (P1 to P4)) in vitro, cell surface marker phenotypes from young cell populations were compared with aged parental synovial fluid-derived MSCs (SF-MSCs) and MSCs at late cell passages (at or after P5) in vitro. Prior studies had identified potential MSC aging-associated cell surface markers using bulk RNA-seq showing at least a two-fold difference in expression levels between iPSC-MSCs and SF-MSCs were determined as identified in Table B:

TABLE B

| | SFMSC | iPSCMSC | Fold |
|---|---|---|---|
| CD28 | 0.45 | 0.18 | 2.45 |
| FCGR2A | 3.09 | 1.37 | 2.25 |
| CD8A | 0.5 | 0.18 | 2.78 |
| CD8B | 0.007 | 0.002 | 3.7 |
| CD6 | 1.27 | 2.99 | 2.35 |
| ICAM2 | 4.38 | 1.8 | 2.44 |
| EPCAM | 1.84 | 5.57 | 3.03 |
| MYOCD | 4.77 | 2.09 | 2.29 |
| SUSD2 | 9.74 | 4.53 | 2.15 |

Quantitative real time-polymerase chain reaction (RT-PCR) analysis was then performed and comparisons of mRNA expression levels in iPSC-MSCs and SF-MSCs for these nine cell surface markers are shown in FIG. 1, wherein seven were upregulated in SF-MSCs (and thus were associated to cell aging) while two were upregulated in iPSC-MSCs (and thus associated with a rejuvenated, younger phenotype).

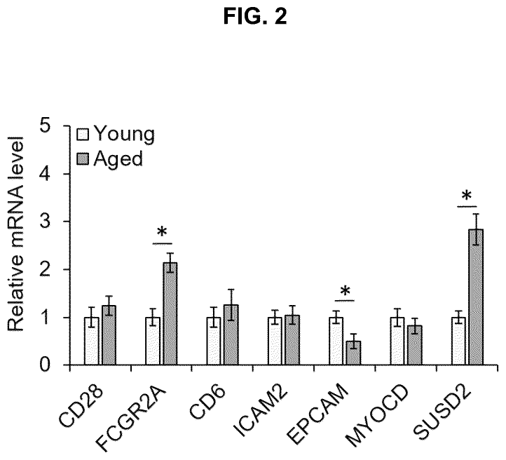

Two of these potential markers (CD8A and CD8B) showed insufficiently informative differences in the expression of mRNA in further analysis of RT-PCR. The other seven cell surface markers were analyzed by expression level comparison between young and aged human SF-MSCs using RT-PCR. The results of these experiments are shown in FIG. 2, demonstrating that PCGR2A and SUSD2 were significantly upregulated while EPCAM was significantly downregulated in aged SF-MSCs compared to those in younger cells to be markers for aged SF-MSCs (thus avoiding any uncertainties in RT-PCR analysis from comparisons between aged SF-MSCs and iPSC-MSCs).

### Example 2

### GATA6 Loss-of-Function Analyses

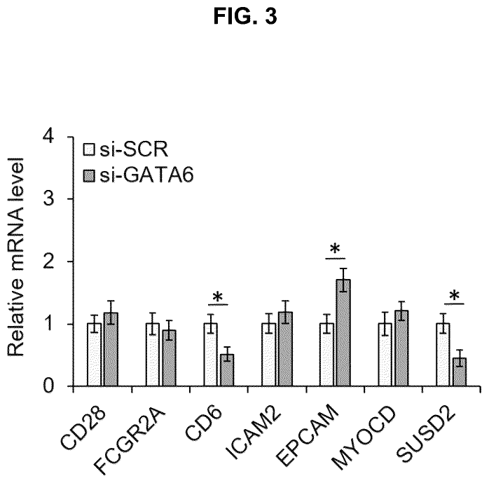

To further evaluate the association between differential expression of these cell surface markers and MSC aging, a “loss-of-function” assay was performed regarding GATA6, an MSC aging-associated transcription factor regulator. If the cell surface markers detected in RNA-seq and RT-PCR assays described in Example 1 were reliably associated with MSC aging then reducing GATA6 expression was expected to effect the differential expression of such markers. GATA6 expression was reduced in SF-MSCs using small interfering RNAs (siRNA) species and the effect of reducing GATA6 expression in these cells is shown in FIG. 3 (compared with the effect on cells into which was reduced a “scrambled” siRNA (siSCR) not specific for GATA6 expression). These results showed that CD6, SUSD2, and EPCAM showed significant differences in expression in SF-MSCs having expression reduced by GATA6-specific siRNA compared with siSCR SF-MSCs. Due to the strength of the statistical association and the differential response revealed in both FIG. 2 and FIG. 3, EPCAM (which showed increased expression in young SF-MSCs) and SUSD2 (which showed increased expression in aged SF-MSCs) were chosen as SF-MSC aging-associated markers.

### Example 3

### Status of Cell Cycle-Associated Gene Expression in Mouse Periosteal MSCs

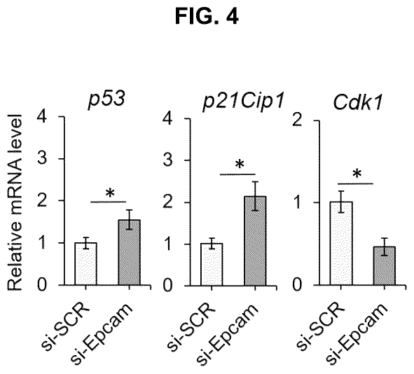

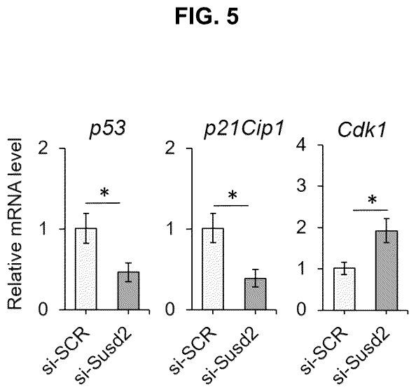

Expression of certain genes are known to be associated with cellular aging; these include p53, p21^{Cip1} and cdk1. Aging mouse periosteal MSCs were expected to have increased expression of p53 and p21^{Cip1} and decreased expression of cdk1. To determine whether loss-of-function of Epcam or Susd2 affected expression of these cell senescence markers, their expression was reduced in MSCs using specific siRNAs compared with cells comprising scrambled siRNAs. These results are shown in FIG. 4 and FIG. 5. FIG. 4 demonstrates cells having siRNA-induced reduction in EPCAM expression showed increased expression of p53 and p21^{Cip1} and decreased expression of cdk1, whereas in FIG. 5 cells having siRNA-induced reduction in SUSD2 expression showed decreased expression of p53 and p21^{Cip1} and increased expression of cdk1. These results provided a demonstration that EPCAM can be used as an “anti-aging” marker and SUSD2 as a “pro-aging” marker.

### Example 4

### Selection of Aged and Young/Juvenile MSCs Based on Cell Surface Markers

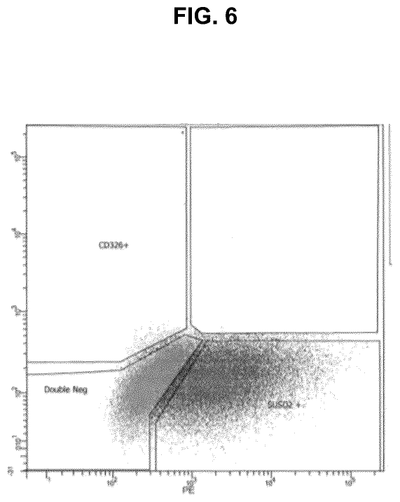

The results shown in Example 3 provided markers for differentially selecting aged and juvenile MSCs from heterogeneous populations of donor MSCs hereof based on cell surface marker expression. To demonstrate the utility of EPCAM and SUSD2 in such cellular age-dependent differential selection, bone marrow-derived MSCs (BM-MSCs) were labeled with antibodies specific for EPCAM (APC antihuman CD326 (EpCAM) Antibody Clone 9C4, obtained from BioLegend, Cat. #324207) and SUSD2 (PE antihuman SUSD2 Antibody Clone W5C5, obtained from Miltenyo Biotech, Cat. #130-117-794) wherein the antibodies were conjugated with fluorescent dyes capable of distinguishing EPCAM- and SUSD2-expressing BM-MSCs by flow cytometry. The results of these experiments are shown in FIG. 6, which demonstrated that 0.62% of BM-MSCs expressed EPCAM only, 48% of BM-MSCs expressed SUSD2 only, 0.78% of BM-MSCs expressed both EPCAM and SUSD2, and 51% of BM-MSCs expressed neither EPCAM nor SUSD2. These results illustrated that while almost half of BM-MSCs expressed SUSD2, less than 1% expressed EPCAM. The EPCAM results disqualified this cell surface marker from being able to distinguish and separate juvenile MSCs, whereas in contrast robust expression of SUSD2 provided a useful marker for aged BM-MSCs.

### Example 5

### Cellular Senescence Detection in Culture-Induced Aging MSCs

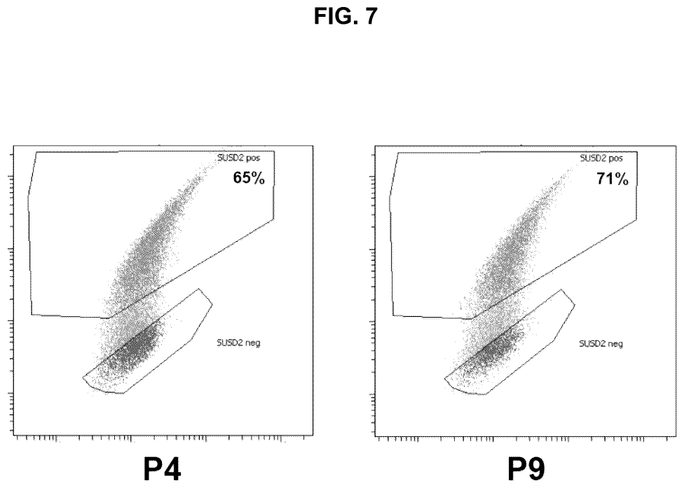

MSCs grown in culture are under environmental stresses, thus undergoing cellular senescence. Whether MSCs in cell culture underwent aging or senescence was determined by comparing SUSD2 expression in cells at low passage numbers such as P4 with cells at a higher passage numbers such as P9. BM-MSCs from cultures at these two passage numbers were labeled with a fluorescently labeled SUSD2 antibody and separated by cytometry on the basis of fluorescence intensity. The results of these experiments are shown in FIG. 7, which shows that 35% of P4 MSCs did not express SUSD2 (were SUSD2−) while the remaining 65% were SUSD2+; in contrast, 29% of P9 MSCs were SUSD2− and the rest 71% were SUSD2+. These results established that SUSD can be used to distinguish cellular aging and senescence of BM-MSCs in culture.

### Example 6

### Senescence-Related Gene Expression in BM-MSCs Sorted Based on SUSD2 Expression

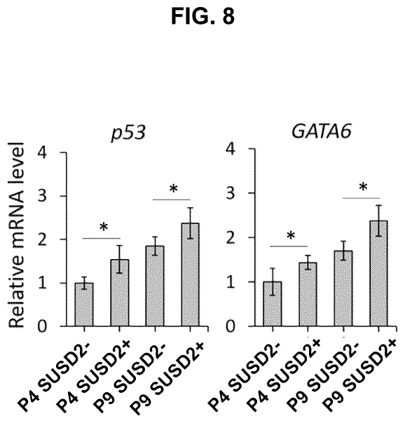

To further establish the correlation between SUSD2-expressing BM-MSCs and the aging/senescence phenotype, p53 and GATA6 expression in SUSD2-expressing cells sorted by flow cytometry was determined. In these experiments, BM-MSCs were sorted from P4 and P9 cell culture and populations of P4/SUSD2−, P4/SUSD2+, P9/SUSD2−, and P9/SUSD2+BM-MSCs were obtained. RT-PCR was then performed for p53 and GATA6 expression on each sorted BM-MSC population. These results are shown in FIG. 8, wherein P9/SUSD2− and P9/SUSD2+ MSCs were found to express higher levels of p53 and GATA6 than P4/SUSD2− and P4/SUSD2+ MSCs. In either P4 or P9 MSCs, greater expression levels of p53 and GATA6 were found in SUSD2+ cells than in SUSD2− cells. These results confirmed that SUSD2 can reliably be used to separate BM-MSCs that express an aged cell population as characterized by p53 and GATA6 senescent cell marker expression.

### Example 7

### Osteogenic and Adipogenic Differentiation in SUSD2-Sorted BM-MSCs

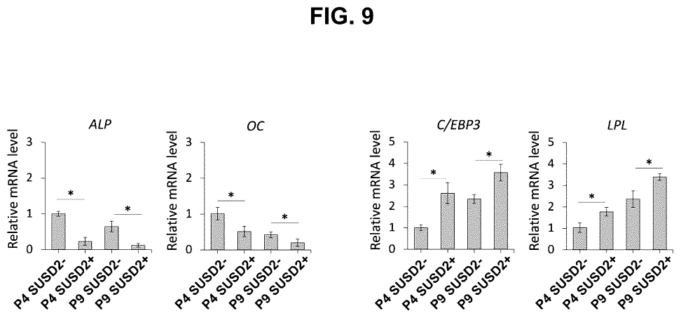

Aged MSCs tend toward adipogenic differentiation and away from osteogenic differentiation, resulting in increased fat and decreased bone formation in such cells. The differentiation propensity of MSCs is also associated with aging of MSCs, wherein juvenile MSCs are pro-osteogenic, leaning toward the differentiation into osteoblasts expressing the bone-associated marker osteocalcin (OC) and aged or senescent MSCs are pro-adipogenic, leaning toward the differentiation into adipocytes expressing the fat-associated marker lipoprotein lipase (LPL). The assay helps determine if the cell is young or aged. Sorted SUSD2− or SUSD2+ BM-MSCs from P4 or P9 cultures were induced to undergo osteogenic and adipogenic differentiation for 21 days. Thereafter, expression levels of OC and LPL were analyzed by RT-PCR. These results are shown in FIG. 9, wherein is shown that for both P4 and P9 MSCs, SUSD2+ cells were more pro-adipogenic than SUSD2− cells. The trend of osteogenic capability among the four sorted MSC populations was opposite of that of adipogenic capability. These results showed that BM-MSCs sorted on the basis of cell surface SUSD2 expression evinced phenotypic differences consistent with SUSD2 being an effective cell surface marker for aging or senescence in BM-MSCs. Accordingly, these results established that SUSD2 can be used as a cell surface marker capable of supporting cell sorting methods including FACS and MACS that separate aged or senescent MSCs from juvenile MSCs.

All publications, patents, and patent applications mentioned in this specification are herein incorporated by reference to the same extent as if each individual publication, patent, and patent application was specifically and individually indicated to be incorporated by reference.

While some embodiments have been illustrated and described in detail in the appended drawings and the foregoing description, such illustration and description are to be considered illustrative and not restrictive. Other variations to the disclosed embodiments can be understood and effected in practicing the claims, from a study of the drawings, the disclosure, and the appended claims. The mere fact that certain measures or features are recited in mutually different dependent claims does not indicate that the combination of these measures or features cannot be used. Any reference signs in the claims should not be construed as limiting the scope.

1. A method for separating robust mesenchymal stem cells for use in stem cell therapies from a population of mesenchymal stem cells of variable robustness, the method comprising:
sorting the cells based on cell surface expression of SUSD2,
wherein cells expressing SUSD2 are separated from cells that do not detectably express SUSD2, and
wherein the population of robust mesenchymal stem cells does not detectably express SUSD2.

2. The method of claim 1 wherein the cells are contacted with a specific binding agent for SUSD2.

3. The method of claim 2, wherein the specific binding agent is an antibody or antigen-binding fragment thereof that specifically binds SUSD2.

4. The method of claim 1, wherein the cells are further sorted for cell surface expression of EPCAM, wherein the population of robust mesenchymal stem cells express EPCAM.

5. The method of claim 4, wherein the cells are contacted with an antibody or antigen-binding fragment thereof that specifically binds EPCAM.

6. The method of claim 5, wherein the antibody or antigen-binding fragment thereof that specifically binds EPCAM is labeled.

7. The method of claim 6, wherein the antibody or antigen-binding fragment thereof specific for binding EPCAM is labeled with a different label than for the antibody or antigen-binding fragment thereof specific for binding SUSD2.

8. The method of claim 7, wherein the cells are sorted for SUSD2 cell surface expression and EPCAM cell surface expression sequentially.

9. The method of claim 7, wherein the cells are sorted for SUSD2 and EPCAM cell surface expression simultaneously.

10. The method of claim 3, wherein the label is a magnetic label.

11. The method of claim 3 wherein the cells are sorted by magnetic cell sorting.

12. An isolated population of robust mesenchymal stem cells, using a method wherein the robust mesenchymal stem cells are separated from a population of mesenchymal stem cells of variable robustness, the method comprising
sorting the cells based on SUSD2 expression
wherein the population does not detectably express SUSD2.

13. An isolated population of robust mesenchymal stem cells, wherein the cells comprising the population produced using the method of claim 4 that express EPCAM do not detectably express SUSD2.

14. A method for enriching from a population of mesenchymal stem cells those cells expressing EPCAM, the method comprising contacting the population of cells with a specific binding agent for EPCAM and separating the cells based on EPCAM expression.

15. The method of claim 14, wherein the specific binding agent is an antibody or antigen-binding fragment thereof that specifically binds EPCAM.

16. The method of claim 14, wherein the enriched cells are further contacted with a labeled specific binding agent that binds to SUSD2 and separated SUSD2 expressing cells.

17. The method of claim 14, wherein the label is a magnetic label and the cells are separated by magnetic cell sorting.

18. (canceled)

19. A method for enriching a population of mesenchymal stem cells based on SUSD2 expression, the method comprising contacting the population of cells with a labeled SUSD2 binding agent and separating the cells based on SUSD2-cell expression.

20. (canceled)

21. The method of claim 19, wherein the label is a magnetic label and the cells are separated by magnetic cell sorting.

22. (canceled)

23. (canceled)

24. A method for administering the population of robust mesenchymal stem cells of claim 12 to a patient in need thereof, wherein the population is formulated in an appropriate carrier.

25-29. (canceled)

25. (canceled)

26. (canceled)


SEQUENCE LISTING (4 sequences)

SEQ ID NO: 1 - DNA, 37 bp, synthetic construct
  source 1..37: mol_type = other DNA
ggtttttgagggtgagggtgagggtgagggtgagggt

SEQ ID NO: 2 - DNA, 39 bp, synthetic construct
  source 1..39: mol_type = other DNA
tcccgactatccctatccctatccctatccctatcccta

SEQ ID NO: 3 - DNA, 23 bp, synthetic construct
  source 1..23: mol_type = other DNA
cagcaagtgggaaggtgtaatcc

SEQ ID NO: 4 - DNA, 25 bp, synthetic construct
  source 1..25: mol_type = other DNA
cccattctatcatcaacgggtacaa